(19) United States
(12) Patent Application Publication
(10) Pub. No.: US 2023/0098944 A1
(43) Pub. Date: Mar. 30, 2023

(54) METHOD OF DECREASING AMYLOID BETA MONOMER LEVELS IN PATIENTS WITH COGNITIVE DECLINE

(71) Applicant: Cognition Therapeutics, Inc., Pittsburgh, PA (US)

(72) Inventors: Susan CATALANO, Pittsburgh, PA (US); Nicholas IZZO, Pittsburgh, PA (US)

(73) Assignee: Cognition Therapeutics, Inc., Pittsburgh, PA (US)

(21) Appl. No.: 17/798,667

(22) PCT Filed: Feb. 12, 2021
(86) PCT No.: PCT/US2021/018023

§ 371 Date: Aug. 10, 2022

Related U.S. Application Data
(60) Provisional application No. 62/976,325, filed on Feb. 13, 2020.

(51) Int. Cl. A61K 31/4035; A61P 25/28

(52) U.S. Cl. CPC A61K 31/4035; A61P 25/28

(57) ABSTRACT

The present disclosure relates to methods of decreasing amyloid β monomers and methods of treating cognitive decline in a subject, wherein the treatment results in an increase in amyloid β oligomers and a reduction in cognitive decline. The present disclosure relates to treating cognitive decline, wherein target engagement can be measured in the clinic after treating.

This application claims the benefit of priority to U.S. Provisional Application No. 62/976,325 entitled “Method of Decreasing Amyloid Beta Monomer Levels in Patients with Cognitive Decline,” filed Feb. 13, 2020, which is incorporated herein by reference in its entirety.

### GOVERNMENT INTERESTS

This invention was made with government support under R43NS083175 awarded by National Institute of Neurological Disorders and Stroke and R43AG037337, R44AG055247, R01AG051593, R01AG054176, R01AG057780, and R01AG058660 awarded by National Institute on Aging. The government has certain rights in the invention.

### SUMMARY OF THE INVENTION

Summary of the Invention: The present disclosure relates to methods of decreasing amyloid β monomers and methods of treating cognitive decline in a subject, wherein the treatment results in an increase in amyloid β oligomers and a reduction in cognitive decline. The present disclosure relates to treating cognitive decline, wherein target engagement can be measured in the clinic after treating.

### DESCRIPTION OF THE DRAWINGS

FIG. 1 describes percent change in levels of Aβ oligomers in patients measured by microimmunoelectrode.

FIG. 2 describes percent change in levels of Aβ oligomers in CSF from patients, measured by Western Blot.

FIG. 3 describes the correlation between measurement of Aβ oligomers measured by microimmunoelectrode and by Western Blot, calculated by Spearman correlation analysis.

FIG. 4 describes CT1812 related increase in Aβ oligomers in patient CSF over time as measured by microimmunoelectrode.

FIG. 5 describes CT1812 related increase in Aβ oligomers in patient CSF over time as measured by Western Blot.

FIG. 6 describes the concentrations of CT1812 in the plasma and CSF of patients after treatment.

FIG. 7 describes a three-point difference in Alzheimer's Disease Assessment Scale (ADAS-COG) score between treated and untreated patients after 185 days of treatment.

FIG. 8 describes the decrease in Aβ monomer (Aβ 1-42) levels in patients treated with a total daily dose of 300 mg CT1812 for 185 days.

### DETAILED DESCRIPTION

### Definitions

Unless defined otherwise, all technical and scientific terms used herein have the same meanings as commonly understood by one of ordinary skill in the art.

The articles “a” and “an” as used herein mean “one or more” or “at least one,” unless otherwise indicated. That is, reference to any element of the present invention by the indefinite article “a” or “an” does not exclude the possibility that more than one of the element is present.

As used herein, the term “about” means plus or minus 10% of the numerical value of the number with which it is being used. Therefore, about 50% means in the range of 45%-55%.

“Administering,” or “administration” and the like, when used in conjunction with the compounds of the disclosure refers to providing the compounds or pharmaceutical compositions according to any of the embodiments described herein, to a subject in need of treatment. Preferably the subject is a mammal, more preferably a human. The present invention comprises administering the compound or pharmaceutical composition of the invention alone or in conjunction with another therapeutic agent. When a compound or pharmaceutical composition of the invention is administered in conjunction with another therapeutic agent, the compound or pharmaceutical composition of the invention and the other therapeutic agent can be administered at the same time or different times, and by the same routes of administration or by different routes of administration. A compound may be administered by oral administration, intravenous administration, intraperitoneal administration, or any other route of administration known in the art.

The term “amyloid β levels” as used herein includes any measurement of the amount of amyloid β measured in a sample collected from a subject. The sample may include, but is not limited to, cerebral spinal fluid, hippocampal interstitial fluid, and plasma. The amyloid β levels may be measured using any method to measure the concentration or amount of a protein, which may include, but it not limited to, use of microimmunoelectrodes or Western Blot.

The term “animal” as used herein includes, but is not limited to, humans and non-human vertebrates such as wild, domestic and farm animals.

As used herein, “cognitive decline” can be any negative change in an animal's cognitive function. For example cognitive decline, includes but is not limited to, memory loss (e.g. behavioral memory loss), failure to acquire new memories, confusion, impaired judgment, personality changes, disorientation, or any combination thereof. A compound that is effective to treat cognitive decline can be thus effective by restoring long term neuronal potentiation (LTP) or long term neuronal depression (LTD) or a balance of synaptic plasticity measured electrophysiologically; inhibiting, treating, and/or abatement of neurodegeneration; inhibiting, treating, and/or abatement of general amyloidosis; inhibiting, treating, abatement of one or more of amyloid production, amyloid assembly, amyloid aggregation, and amyloid oligomer binding; inhibiting, treating, and/or abatement of a nonlethal effect of one or more of Aβ species on a neuron cell (such as synapse loss or dysfunction and abnormal membrane trafficking); and any combination thereof. Additionally, that compound can also be effective in treating Aβ related neurodegenerative diseases and disorders including, but not limited to dementia, including but not limited to Alzheimer's Disease (AD) including mild Alzheimer's disease, Down's syndrome, vascular dementia (cerebral amyloid angiopathy and stroke), dementia with Lewy bodies, HIV dementia, Mild Cognitive Impairment (MCI); Age-Associated Memory Impairment (AAMI); Age-Related Cognitive Decline (ARCD), preclinical Alzheimer's Disease (PCAD); and Cognitive Impairment No Dementia (CIND).

The phrase “pharmaceutically acceptable” refers to those compounds, materials, pharmaceutical compositions, and/or dosage forms that are, within the scope of sound medical judgment, generally regarded as safe and nontoxic. In particular, pharmaceutically acceptable carriers, diluents or other excipients used in the pharmaceutical compositions of this disclosure are physiologically tolerable, compatible with other ingredients, and do not typically produce an allergic or similar untoward reaction (for example, gastric upset, dizziness and the like) when administered to a patient. Preferably, as used herein, the term “pharmaceutically acceptable” means approved by a regulatory agency of the Federal government or a state government or listed in the U.S. Pharmacopoeia or other generally recognized pharmacopoeia for use in animals, and more particularly in humans.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic. The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa., 1990, the disclosure of which is hereby incorporated by reference.

The terms “subject,” “individual” or “patient” are used interchangeably and as used herein are intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients. The methods are particularly suitable for treating human patients having a condition, disease or disorder described herein.

As used herein, the term “therapeutic” means an agent utilized to treat, combat, ameliorate, prevent or improve an unwanted condition or disease of a patient.

A “therapeutically effective amount” of a compound, pharmaceutically acceptable salt thereof or pharmaceutical composition according to any embodiment described herein, is an amount sufficient to produce a selected effect on at least one symptom or parameter of a specific disease or disorder. The therapeutic effect may be objective (i.e., measurable by some test or marker) or subjective (i.e., subject gives an indication of or feels an effect or physician observes a change). The effect contemplated herein, includes both medical therapeutic and/or prophylactic treatment, as appropriate. The specific dose of a compound administered according to this disclosure to obtain therapeutic and/or prophylactic effects is determined by the particular circumstances surrounding the case, including, for example, the compound administered, the route of administration, the co-administration of other active ingredients, the condition being treated, the activity of the specific compound employed, the specific composition employed, the age, body weight, general health, sex and diet of the patient; the time of administration, route of administration, and rate of excretion of the specific compound employed and the duration of the treatment. The therapeutically effective amount administered will be determined by the physician in the light of the foregoing relevant circumstances and the exercise of sound medical judgment. A therapeutically effective amount of a compound, according to any embodiment described herein, is typically an amount such that when it is administered in a physiologically tolerable excipient composition, it is sufficient to achieve an effective systemic concentration or local concentration in the tissue.

The terms “treat,” “treated,” or “treating” as used herein, refers to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this disclosure, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects. Treatment also includes prolonging survival as compared to expected survival if not receiving treatment.

### Methods

The present disclosure relates to clinical results in human, the results of which were surprising and unexpected in light of the information available to a skilled artisan. The animal models and preclinical data suggested that CT1812 can selectively displace oligomers and increase their concentration in cerebral spinal fluid and in brain interstitial fluid but had not effect on the concentration of monomers. This was supported by data in human subjects treated with CT1812 for 28 days, which resulted in no change in amyloid β monomer concentration in the cerebral spinal fluid or interstitial fluid (See 62/976,325, which is included herein by reference). The results presented here are surprising and unexpected as in some embodiments of the present disclosure describe a method of reducing amyloid β in a human subject. In some embodiments of the present disclosure, a method of elevating amyloid β oligomer levels in a human subject is described.

In some embodiments the present disclosure describes a method of reducing amyloid β monomer levels in a subject comprising administering to the subject a therapeutically effective amount of a compound of formula:

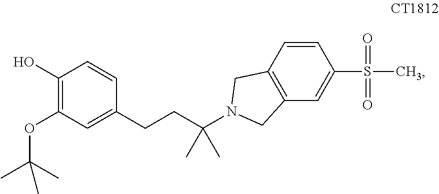 [SMILES: CC(C)(C)Oc1cc(CCC(C)(C)N2Cc3ccc(S(C)(=O)=O)cc3C2)ccc1O]

or a pharmaceutically acceptable salt thereof.

In some embodiments described herein, the subject is a human. In some embodiments described herein, the subject is a subject with cognitive decline. In some embodiments described herein, the subject is a subject with cognitive decline wherein the cognitive decline is Alzheimer's disease. In some embodiments described herein, the subject is a subject with Alzheimer's disease.

In some embodiments the compound is administered orally. In some embodiments, the compound is administered by a route selected from the group comprising oral administration, intravenous administration, intramuscular administration, subcutaneous administration, intranasal administration, intradermal administration, topical administration, In some embodiments, the compound may be formulated as a tablet, as a capsule, as a powder, in a solution, in a suspension, and in an emulsion,

In some embodiments a total daily dose of 10 mg to 2000 mg, of the compound is administered. The total daily dose of the compounds of this disclosure can be administered to a subject in single dose or in divided doses. In some embodiments the dose is 100 mg per day, in some embodiments the dose is 300 mg per day, in some embodiments the dose is 560 mg. In some embodiments a total daily dose of 100 mg of the compound is administered for at least about 6 months. In some embodiments a total daily dose of 300 mg of the compound is administered for at least about 6 months. In some embodiments a total daily dose of 560 mg is administered for at least about 6 months. In some embodiments the total daily does is about 10 mg to about 2000 mg, about 100 mg to about 2000 mg, about 200 mg to about 2000 mg, about 300 mg to about 2000 mg, about 400 mg to about 2000 mg, about 500 mg to about 2000 mg, about 600 mg to about 2000 mg, about 700 mg to about 2000 mg, about 800 mg to about 2000 mg, about 900 mg to about 2000 mg, about 1000 mg to about 2000 mg, about 1200 mg to about 2000 mg, about 1400 mg to about 2000 mg, about 1600 mg to about 2000 mg, about 1800 mg to about 2000 mg, or a value within these ranges. Specific examples may include about 50 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, or a range between any two of these values.

In some embodiments CT1812 is administered for at least 6 months. In some embodiments, some embodiments CT1812 is administered for about 1 day to about 50 years. In some embodiments CT1812 is administered for about 1 day to about 1 year. In some embodiments CT1812 is administered for about 1 day to about 6 months, about 3 days to about 6 months, about 1 week to about 6 months, about 2 weeks to about 6 months, about 3 weeks to about 6 months, about 1 month to about 6 months, about 2 months to about 6 months, about 3 months to about 6 months, about 4 months to about 6 months, about 5 months to about 6 months, or a value within these ranges. Specific examples may include about 1 day, about 2 days, about 3 days, about 4 days, about 5 days, about 6 days, about 7 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 1 year or a range between any two of these values.

In some embodiments the administration of CT1812 results in a change in amyloid β levels in the cerebral spinal fluid (CSF), hippocampal interstitial fluid, plasma, or a combination thereof. In some embodiments the administration of CT1812 results in a reduction of amyloid β monomer levels. In some embodiments, the amyloid β monomer is selected from Aβ 1-40 and Aβ 1-42. In some embodiments the administration of CT1812 results in an elevation of amyloid β oligomers in the cerebral spinal fluid (CSF), hippocampal interstitial fluid, plasma, or a combination thereof. In some embodiments the level of amyloid β after administration of CT1812 is relative to the level of amyloid β before administration of CT1812. In some embodiments, the amyloid β levels are measured using microimmunoelectrodes. In some embodiments, the amyloid β levels are measured using Western Blot. In some embodiments, the amyloid β levels are measured using ELISA (enzyme-linked immunosorbent assay).

In some embodiments, the administration of CT1812 results in a reduction in cognitive decline. In some embodiments, the administration of CT1812 blocks cognitive decline. In some embodiments, the administration of CT1812 results in a reduction in the Alzheimer's Disease Assessment Score (ADAS-COG) score of a subject as compared to the ADAS-COG score of the subject before treatment. In some embodiments the administration of CT1812 results in a subject maintaining the Alzheimer's Disease Assessment Score (ADAS-COG) score of a subject as compared to the ADAS-COG score of the subject before treatment. In some embodiments, the administration of CT1812 results in an at least 3 point reduction in the Alzheimer's Disease Assessment Score (ADAS-COG) score of a subject as compared to the ADAS-COG score of the subject before treatment.

In some embodiments, the present disclosure describes a method of reducing amyloid β monomer levels in a subject comprising administering to the subject a therapeutically effective amount of a pharmaceutical composition comprising a compound of formula:

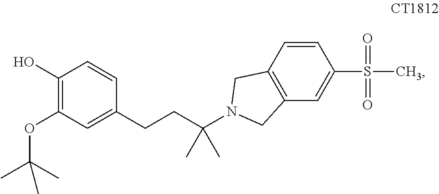 [SMILES: CC(C)(C)Oc1cc(CCC(C)(C)N2Cc3ccc(S(C)(=O)=O)cc3C2)ccc1O]

or a pharmaceutically acceptable salt thereof,

wherein the pharmaceutical composition comprises at least one pharmaceutically acceptable excipient.

In some embodiments, the subject is a human. In some embodiments described herein, the subject is a subject with cognitive decline. In some embodiments described herein, the subject is a subject with cognitive decline wherein the cognitive decline is Alzheimer's disease. In some embodiments described herein, the subject is a subject with Alzheimer's disease.

In some embodiments the compound is administered orally. In some embodiments, the compound is administered by a route selected from the group comprising oral administration, intravenous administration, intramuscular administration, subcutaneous administration, intranasal administration, intradermal administration, topical administration, In some embodiments, the compound may be formulated as a tablet, as a capsule, as a powder, in a solution, in a suspension, and in an emulsion,

In some embodiments a total daily dose of 10 mg to 2000 mg, of the compound in a pharmaceutical composition is administered. The total daily dose of the compounds of this disclosure can be administered to a subject in single dose or in divided doses. In some embodiments the dose of the compound in a pharmaceutical composition is 100 mg per day, in some embodiments the dose of the compound in a pharmaceutical composition is 300 mg per day, in some embodiments the dose of the compound in a pharmaceutical composition is 560 mg per day. In some embodiments a total daily dose of 100 mg of the compound in a pharmaceutical composition is administered for at least about 6 months. In some embodiments a total daily dose of 300 mg of the compound in a pharmaceutical composition is administered for at least about 6 months. In some embodiments a total daily dose of about 560 mg of the compound in a pharmaceutical composition is administered for at least about 6 months. In some embodiments the total daily dose is about 10 mg to about 2000 mg, about 100 mg to about 2000 mg, about 200 mg to about 2000 mg, about 300 mg to about 2000 mg, about 400 mg to about 2000 mg, about 500 mg to about 2000 mg, about 600 mg to about 2000 mg, about 700 mg to about 2000 mg, about 800 mg to about 2000 mg, about 900 mg to about 2000 mg, about 1000 mg to about 2000 mg, about 1200 mg to about 2000 mg, about 1400 mg to about 2000 mg, about 1600 mg to about 2000 mg, about 1800 mg to about 2000 mg, or a value within these ranges. Specific examples may include about 50 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, or a range between any two of these values.

In some embodiments the pharmaceutical composition is administered for at least 6 months. In some embodiments, some embodiments the pharmaceutical composition is administered for about 1 day to about 50 years. In some embodiments the pharmaceutical composition is administered for about 1 day to about 1 year. In some embodiments the pharmaceutical composition is administered for about 1 day to about 6 months, about 3 days to about 6 months, about 1 week to about 6 months, about 2 weeks to about 6 months, about 3 weeks to about 6 months, about 1 month to about 6 months, about 2 months to about 6 months, about 3 months to about 6 months, about 4 months to about 6 months, about 5 months to about 6 months, or a value within these ranges. Specific examples may include about 1 day, about 2 days, about 3 days, about 4 days, about 5 days, about 6 days, about 7 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 1 year or a range between any two of these values.

In some embodiments the administration of CT1812 results in a change in amyloid β levels in the cerebral spinal fluid (CSF), hippocampal interstitial fluid, plasma, or a combination thereof. In some embodiments the administration of CT1812 results in a reduction of amyloid β monomer levels. In some embodiments, the amyloid β monomer is selected from Aβ 1-40 and Aβ 1-42. In some embodiments the administration of CT1812 results in an elevation of amyloid β oligomers in the cerebral spinal fluid (CSF), hippocampal interstitial fluid, plasma, or a combination thereof. In some embodiments the level of amyloid β after administration of CT1812 is relative to the level of amyloid β before administration of CT1812. In some embodiments, the amyloid β levels are measured using microimmunoelectrodes. In some embodiments, the amyloid β levels are measured using Western Blot. In some embodiments, the amyloid β levels are measured using ELISA (enzyme-linked immunosorbent assay).

In some embodiments, the administration of the pharmaceutical composition results in a reduction in cognitive decline. In some embodiments, the administration of the pharmaceutical composition blocks cognitive decline. In some embodiments, the administration of the pharmaceutical composition results in a reduction in the Alzheimer's Disease Assessment Score (ADAS-COG) score of a subject as compared to the ADAS-COG score of the subject before treatment. In some embodiments the administration of the pharmaceutical composition results in a subject maintaining the Alzheimer's Disease Assessment Score (ADAS-COG) score of a subject as compared to the ADAS-COG score of the subject before treatment. In some embodiments, the administration of the pharmaceutical composition results in an at least 3 point reduction in the Alzheimer's Disease Assessment Score (ADAS-COG) score of a subject as compared to the ADAS-COG score of the subject before treatment.

In some embodiments, the present disclosure describes a method of elevating amyloid β oligomer levels in a subject comprising administering to the subject a therapeutically effective amount of a compound of formula:

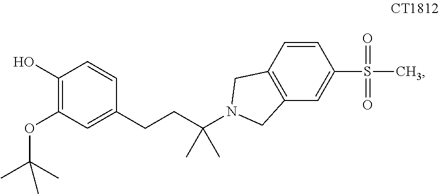 [SMILES: CC(C)(C)Oc1cc(CCC(C)(C)N2Cc3ccc(S(C)(=O)=O)cc3C2)ccc1O]

or a pharmaceutically acceptable salt thereof.

In some embodiments described herein, the subject is a human. In some embodiments described herein, the subject is a subject with cognitive decline. In some embodiments described herein, the subject is a subject with cognitive decline wherein the cognitive decline is Alzheimer's disease. In some embodiments described herein, the subject is a subject with Alzheimer's disease.

In some embodiments the compound is administered orally. In some embodiments, the compound is administered by a route selected from the group comprising oral administration, intravenous administration, intramuscular administration, subcutaneous administration, intranasal administration, intradermal administration, topical administration, In some embodiments, the compound may be formulated as a tablet, as a capsule, as a powder, in a solution, in a suspension, and in an emulsion,

In some embodiments a total daily dose of 10 mg to 2000 mg, of the compound is administered. The total daily dose of the compounds of this disclosure can be administered to a subject in single dose or in divided doses. In some embodiments the dose is 100 mg per day, in some embodiments the dose is 300 mg per day, in some embodiments the dose is about 560 mg per day. In some embodiments a total daily dose of 100 mg of the compound is administered one time. In some embodiments a total daily dose of 300 mg of the compound is administered one time. In some embodiments a total daily dose of 560 mg of the compound is administered one time. In some embodiments the total daily does is about 10 mg to about 2000 mg, about 100 mg to about 2000 mg, about 200 mg to about 2000 mg, about 300 mg to about 2000 mg, about 400 mg to about 2000 mg, about 500 mg to about 2000 mg, about 600 mg to about 2000 mg, about 700 mg to about 2000 mg, about 800 mg to about 2000 mg, about 900 mg to about 2000 mg, about 1000 mg to about 2000 mg, about 1200 mg to about 2000 mg, about 1400 mg to about 2000 mg, about 1600 mg to about 2000 mg, about 1800 mg to about 2000 mg, or a value within these ranges. Specific examples may include about 50 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, or a range between any two of these values.

In some embodiments CT1812 is administered one time. In some embodiments CT1812 is administered for at least 6 months. In some embodiments, CT1812 is administered for about 1 day to about 50 years. In some embodiments, CT1812 is administered for about 1 day to about 1 year. In some embodiments, CT1812 is administered for about 1 day to about 6 months, about 3 days to about 6 months, about 1 week to about 6 months, about 2 weeks to about 6 months, about 3 weeks to about 6 months, about 1 month to about 6 months, about 2 months to about 6 months, about 3 months to about 6 months, about 4 months to about 6 months, about 5 months to about 6 months, or a value within these ranges. Specific examples may include about 1 day, about 2 days, about 3 days, about 4 days, about 5 days, about 6 days, about 7 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 1 year or a range between any two of these values.

In some embodiments the amyloid β oligomers levels are measured within 24 hours of CT1812 administration. In some embodiments the amyloid β oligomer levels are measured within 1 week of CT1812 administration. In some embodiments the amyloid β oligomers levels are measured within 1 month of CT1812 administration. In some embodiments the amyloid β oligomers levels are measured within 6 months of CT1812 administration. In some embodiments the amyloid β oligomers levels are measured at a time point from about 15 minutes to about 12 months after CT1812 administration, about 15 minutes to about 24 hours after CT1812 administration, about 30 minutes to about 24 hours after CT1812 administration, about 45 minutes to about 24 hours after CT1812 administration, about 1 hour to about 24 hours after CT1812 administration, about 2 hours to about 24 hours after CT1812 administration, about 3 hours to about 24 hours after CT1812 administration, about 4 hours to about 24 hours after CT1812 administration, about 6 hours to about 24 hours after CT1812 administration, about 8 hours to about 24 hours after CT1812 administration, about 10 hours to about 24 hours after CT1812 administration, about 12 hours to about 24 hours after CT1812 administration, about 16 hours to about 24 hours after CT1812 administration, about 20 hours to about 24 hours after CT1812 administration, about 1 day to about 7 days after CT1812 administration, about 2 days to about 7 days after CT1812 administration, about 1 week to about 4 weeks after CT1812 administration, about 2 weeks to about 4 weeks after CT1812 administration, about 3 weeks to about 4 weeks after CT1812 administration, about 1 month to about 12 months after CT1812 administration, about 2 months to about 12 months after CT1812 administration, about 3 months to about 12 months after CT1812 administration, about 4 months to about 12 months after CT1812 administration, about 5 months to about 12 months after CT1812 administration, about 6 months to about 12 months after CT1812 administration, about 7 months to about 12 months after CT1812 administration, about 8 months to about 12 months after CT1812 administration, about 9 months to about 12 months after CT1812 administration, about 10 months to about 12 months after CT1812 administration, about 11 months to about 12 months after CT1812 administration.

In some embodiments, the amyloid β levels are indicative of target engagement after administration of CT1812. In some embodiments, the amyloid β levels increase after administration of CT1812, wherein the increase in amyloid β levels is indicative of target engagement.

In some embodiments the administration of CT1812 results in a change in amyloid β levels in the cerebral spinal fluid (CSF), hippocampal interstitial fluid, plasma, or a combination thereof. In some embodiments the administration of CT1812 results in a reduction of amyloid β monomer levels. In some embodiments, the amyloid β monomer is selected from Aβ 1-40 and Aβ 1-42. In some embodiments the administration of CT1812 results in an elevation of amyloid β oligomers in the cerebral spinal fluid (CSF), hippocampal interstitial fluid, plasma, or a combination thereof. In some embodiments the level of amyloid β after administration of CT1812 is relative to the level of amyloid β before administration of CT1812. In some embodiments, the amyloid β levels are measured using micorimmunoelectrodes. In some embodiments, the amyloid β levels are measured using Western Blot. In some embodiments, the amyloid β levels are measured using ELISA (enzyme-linked immunosorbent assay).

In some embodiments, the administration of CT1812 results in a reduction in cognitive decline. In some embodiments, the administration of CT1812 blocks cognitive decline. In some embodiments, the administration of CT1812 results in a reduction in the Alzheimer's Disease Assessment Score (ADAS-COG) score of a subject as compared to the ADAS-COG score of the subject before treatment. In some embodiments the administration of CT1812 results in a subject maintaining the Alzheimer's Disease Assessment Score (ADAS-COG) score of a subject as compared to the ADAS-COG score of the subject before treatment. In some embodiments, the administration of CT1812 results in an at least 3 point reduction in the Alzheimer's Disease Assessment Score (ADAS-COG) score of a subject as compared to the ADAS-COG score of the subject before treatment.

In some embodiments, the present disclosure describes a method of treating cognitive decline in a subject comprising administering to the subject a therapeutically effective amount of a compound of formula:

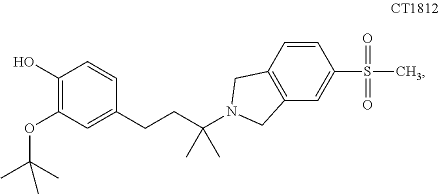 [SMILES: CC(C)(C)Oc1cc(CCC(C)(C)N2Cc3ccc(S(C)(=O)=O)cc3C2)ccc1O]

or a pharmaceutically acceptable salt thereof.

In some embodiments described herein, the subject is a human. In some embodiments described herein, the subject is a subject with cognitive decline. In some embodiments described herein, the subject is a subject with cognitive decline wherein the cognitive decline is Alzheimer's disease. In some embodiments described herein, the subject is a subject with Alzheimer's disease.

In some embodiments the compound is administered orally. In some embodiments, the compound is administered by a route selected from the group comprising oral administration, intravenous administration, intramuscular administration, subcutaneous administration, intranasal administration, intradermal administration, topical administration, In some embodiments, the compound may be formulated as a tablet, as a capsule, as a powder, in a solution, in a suspension, and in an emulsion,

In some embodiments a total daily dose of 10 mg to 2000 mg, of the compound is administered. The total daily dose of the compounds of this disclosure can be administered to a subject in single dose or in divided doses. In some embodiments the dose is 100 mg per day, in some embodiments the dose is 300 mg per day, in some embodiments the dose is about 560 mg. In some embodiments a total daily dose of 100 mg of the compound is administered for at least about 6 months. In some embodiments a total daily dose of 300 mg of the compound is administered for at least about 6 months. In some embodiments a total daily dose of about 560 mg of the compound is administered for at least about 6 months. In some embodiments the total daily does is about 10 mg to about 2000 mg, about 100 mg to about 2000 mg, about 200 mg to about 2000 mg, about 300 mg to about 2000 mg, about 400 mg to about 2000 mg, about 500 mg to about 2000 mg, about 600 mg to about 2000 mg, about 700 mg to about 2000 mg, about 800 mg to about 2000 mg, about 900 mg to about 2000 mg, about 1000 mg to about 2000 mg, about 1200 mg to about 2000 mg, about 1400 mg to about 2000 mg, about 1600 mg to about 2000 mg, about 1800 mg to about 2000 mg, or a value within these ranges. Specific examples may include about 50 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, or a range between any two of these values.

In some embodiments CT1812 is administered for at least 6 months. In some embodiments, some embodiments CT1812 is administered for about 1 day to about 50 years. In some embodiments CT1812 is administered for about 1 day to about 1 year. In some embodiments CT1812 is administered for about 1 day to about 6 months, about 3 days to about 6 months, about 1 week to about 6 months, about 2 weeks to about 6 months, about 3 weeks to about 6 months, about 1 month to about 6 months, about 2 months to about 6 months, about 3 months to about 6 months, about 4 months to about 6 months, about 5 months to about 6 months, or a value within these ranges. Specific examples may include about 1 day, about 2 days, about 3 days, about 4 days, about 5 days, about 6 days, about 7 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 1 year or a range between any two of these values.

In some embodiments the administration of CT1812 results in a change in amyloid β levels in the cerebral spinal fluid (CSF), hippocampal interstitial fluid, plasma, or a combination thereof. In some embodiments the administration of CT1812 results in a reduction of amyloid β monomer levels. In some embodiments, the amyloid β monomer is selected from Aβ 1-40 and Aβ 1-42. In some embodiments the administration of CT1812 results in an elevation of amyloid β oligomers in the cerebral spinal fluid (CSF), hippocampal interstitial fluid, plasma, or a combination thereof. In some embodiments the level of amyloid β after administration of CT1812 is relative to the level of amyloid β before administration of CT1812. In some embodiments, the amyloid β levels are measured using microimmunoelectrodes. In some embodiments, the amyloid β levels are measured using Western Blot. In some embodiments, the amyloid β levels are measured using ELISA (enzyme-linked immunosorbent assay).

In some embodiments, the administration of CT1812 results in a reduction in cognitive decline. In some embodiments, the administration of CT1812 blocks cognitive decline. In some embodiments, the administration of CT1812 results in a reduction in the Alzheimer's Disease Assessment Score (ADAS-COG) score of a subject as compared to the ADAS-COG score of the subject before treatment. In some embodiments the administration of CT1812 results in a subject maintaining the Alzheimer's Disease Assessment Score (ADAS-COG) score of a subject as compared to the ADAS-COG score of the subject before treatment. In some embodiments, the administration of CT1812 results in an at least 3 point reduction in the Alzheimer's Disease Assessment Score (ADAS-COG) score of a subject as compared to the ADAS-COG score of the subject before treatment.

In some embodiments, the present disclosure describes method of treating Alzheimer's disease in a subject comprising administering to the subject a therapeutically effective amount of a compound of formula:

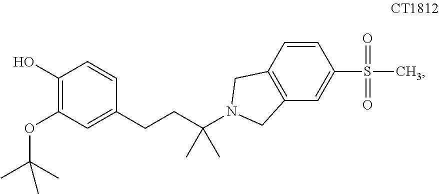 [SMILES: CC(C)(C)Oc1cc(CCC(C)(C)N2Cc3ccc(S(C)(=O)=O)cc3C2)ccc1O]

or a pharmaceutically acceptable salt thereof.

In some embodiments described herein, the subject is a human. In some embodiments described herein, the subject is a subject with cognitive decline. In some embodiments described herein, the subject is a subject with cognitive decline wherein the cognitive decline is Alzheimer's disease. In some embodiments described herein, the subject is a subject with Alzheimer's disease.

In some embodiments the compound is administered orally. In some embodiments, the compound is administered by a route selected from the group comprising oral administration, intravenous administration, intramuscular administration, subcutaneous administration, intranasal administration, intradermal administration, topical administration, In some embodiments, the compound may be formulated as a tablet, as a capsule, as a powder, in a solution, in a suspension, and in an emulsion,

In some embodiments a total daily dose of 10 mg to 2000 mg, of the compound is administered. The total daily dose of the compounds of this disclosure can be administered to a subject in single dose or in divided doses. In some embodiments the dose is 100 mg per day, in some embodiments the dose is 300 mg per day, in some embodiments the dose is about 560 mg. In some embodiments a total daily dose of 100 mg of the compound is administered for at least about 6 months. In some embodiments a total daily dose of 300 mg of the compound is administered for at least about 6 months. In some embodiments a total daily dose of 560 mg of the compound is administered for at least about 6 months. In some embodiments the total daily does is about 10 mg to about 2000 mg, about 100 mg to about 2000 mg, about 200 mg to about 2000 mg, about 300 mg to about 2000 mg, about 400 mg to about 2000 mg, about 500 mg to about 2000 mg, about 600 mg to about 2000 mg, about 700 mg to about 2000 mg, about 800 mg to about 2000 mg, about 900 mg to about 2000 mg, about 1000 mg to about 2000 mg, about 1200 mg to about 2000 mg, about 1400 mg to about 2000 mg, about 1600 mg to about 2000 mg, about 1800 mg to about 2000 mg, or a value within these ranges. Specific examples may include about 50 mg, about 100 mg, about 150 mg, about 200 mg, about 250 mg, about 300 mg, about 350 mg, about 400 mg, about 450 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, or a range between any two of these values.

In some embodiments CT1812 is administered for at least 6 months. In some embodiments, some embodiments CT1812 is administered for about 1 day to about 50 years. In some embodiments CT1812 is administered for about 1 day to about 1 year. In some embodiments CT1812 is administered for about 1 day to about 6 months, about 3 days to about 6 months, about 1 week to about 6 months, about 2 weeks to about 6 months, about 3 weeks to about 6 months, about 1 month to about 6 months, about 2 months to about 6 months, about 3 months to about 6 months, about 4 months to about 6 months, about 5 months to about 6 months, or a value within these ranges. Specific examples may include about 1 day, about 2 days, about 3 days, about 4 days, about 5 days, about 6 days, about 7 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 1 year or a range between any two of these values.

In some embodiments the administration of CT1812 results in a change in amyloid β levels in the cerebral spinal fluid (CSF), hippocampal interstitial fluid, plasma, or a combination thereof. In some embodiments the administration of CT1812 results in a reduction of amyloid β monomer levels. In some embodiments, the amyloid β monomer is selected from Aβ 1-40 and Aβ 1-42. In some embodiments the administration of CT1812 results in an elevation of amyloid β oligomers in the cerebral spinal fluid (CSF), hippocampal interstitial fluid, plasma, or a combination thereof. In some embodiments the level of amyloid β after administration of CT1812 is relative to the level of amyloid β before administration of CT1812. In some embodiments, the amyloid β levels are measured using microimmunoelectrodes. In some embodiments, the amyloid β levels are measured using Western Blot. In some embodiments, the amyloid β levels are measured using ELISA (enzyme-linked immunosorbent assay).

In some embodiments, the administration of CT1812 results in a reduction in cognitive decline. In some embodiments, the administration of CT1812 blocks cognitive decline. In some embodiments, the administration of CT1812 results in a reduction in the Alzheimer's Disease Assessment Score (ADAS-COG) score of a subject as compared to the ADAS-COG score of the subject before treatment. In some embodiments the administration of CT1812 results in a subject maintaining the Alzheimer's Disease Assessment Score (ADAS-COG) score of a subject as compared to the ADAS-COG score of the subject before treatment. In some embodiments, the administration of CT1812 results in an at least 3 point reduction in the Alzheimer's Disease Assessment Score (ADAS-COG) score of a subject as compared to the ADAS-COG score of the subject before treatment.

### EXAMPLES

### Example 1: Clinical Efficacy of CT1812

Methods: A Phase 2 clinical trial (NCT03522129) was conducted enrolling patients with mild-to-moderate Alzheimer's disease (MMSE 18-26) into the Randomized, double-blind, placebo-controlled, single dose administration of CT1812 followed by hourly sampling of lumbar CSF via indwelling catheter for 24 hours. The primary objective of the study was to evaluate target engagement of CT1812 treatment by measuring the displacement of Aβ oligomers into cerebrospinal fluid (CSF). The was quantified by a measuring change from the baseline CSF Aβ oligomer concentration after dosing with CT1812 versus placebo. Microimmunoelectrodes (Yuede et al 2016) coated with oligomer-specific antibody, A11 were placed in CSF samples of Alzheimer's disease patient samples to detect soluble Aβ after a single dose of CT1812 (560 mg) or placebo.

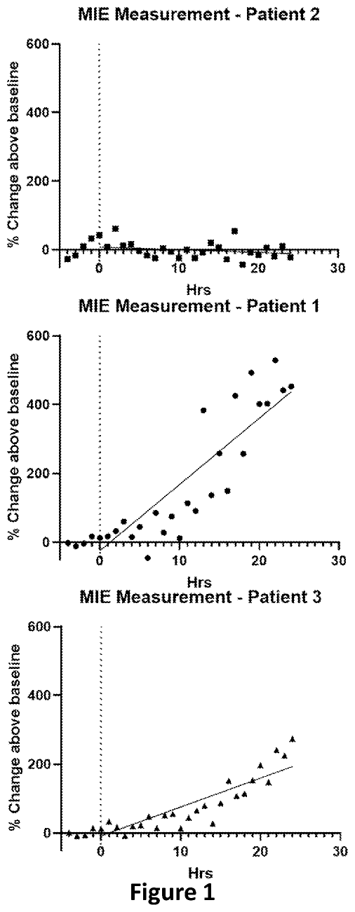

Results: Microimmunoelectrode (MIE) measurements of Aβ oligomers in CSF show a drug-dependent rise in Aβ oligomers over time. After a pre-dose baseline was established, MIE measurements of oligomer levels showed an apparent increase with time in the CSF of patients receiving a single dose of CT1812 (vertical dashed line) but not in patient receiving placebo (FIG. 1). The slopes for Patients 1 and 3 were significantly non-zero [p<0.001 for each patient, R²=0.7720 (F=91.45) and R²=0.7898 (F=101.5), respectively while the linear slope for the placebo-treated patient (Patient 2) was not different from zero (p=0.6849, R2=0.06891 F=1.998.

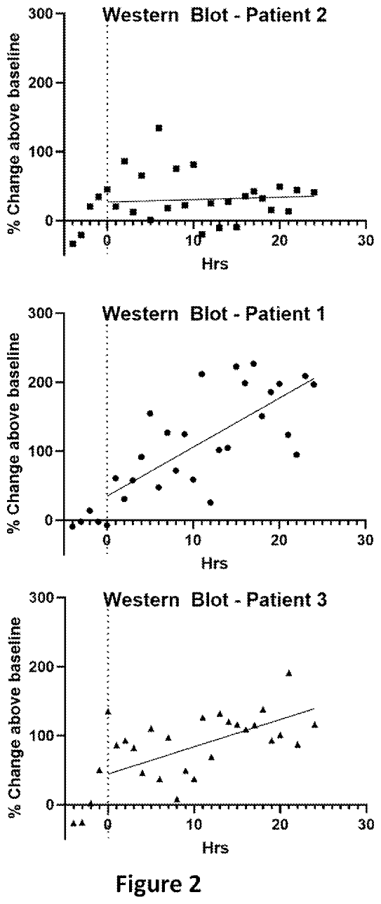

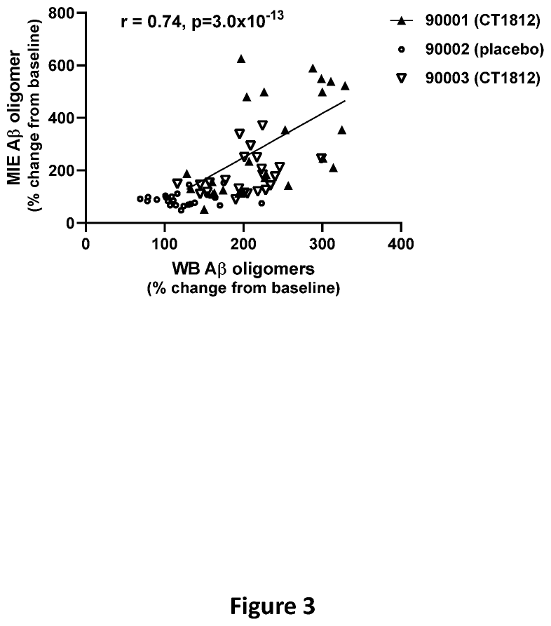

MIE measurements were confirmed using Western Blot to measure protein levels. Alzheimer's disease patient CSF samples (N=3 patients) were run on native Western Blot probed with AB specific antibody, 82E1. Following the single dose of CT1812 (vertical dashed line), Oligomer levels showed an apparent increase with time in the CSF of patients receiving a single dose of CT1812 (vertical dashed line) but not in patient receiving placebo (FIG. 2). The slopes for Patients 1 and 3 were significantly non-zero [p<0.001 for each patient, R²=0.9808 (F=41.91) and R2=0.3997 (F=17.31), respectively] while the linear slope for the placebo-treated patient (Patient 2) was not different from zero (p=0.0.6849, R²=0.006437 F=0.1684). Spearman correlation analysis shows significant correlation between MIE and western blot oligomer measurements (FIG. 3, r—0.74, p=3×10⁻¹³).

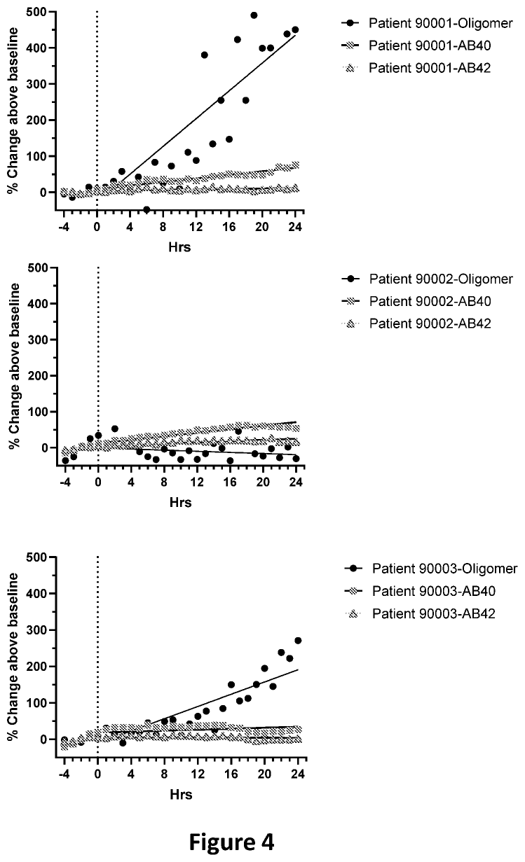

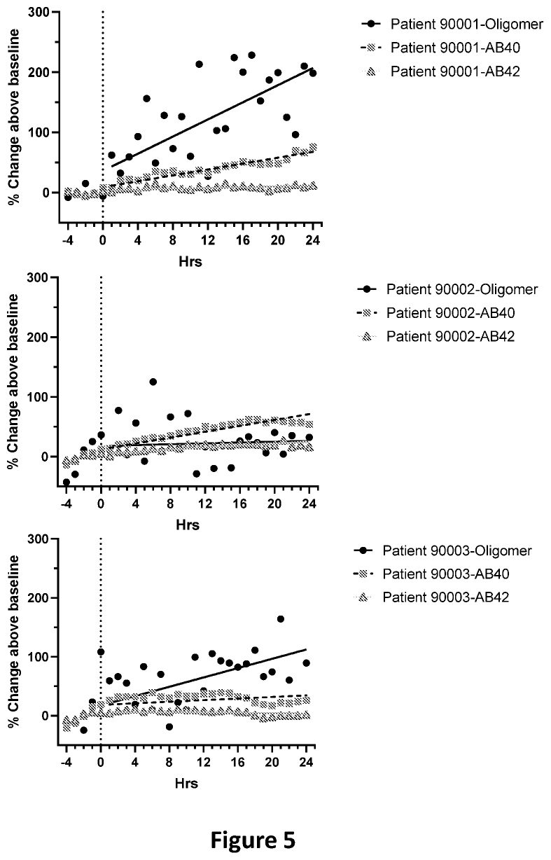

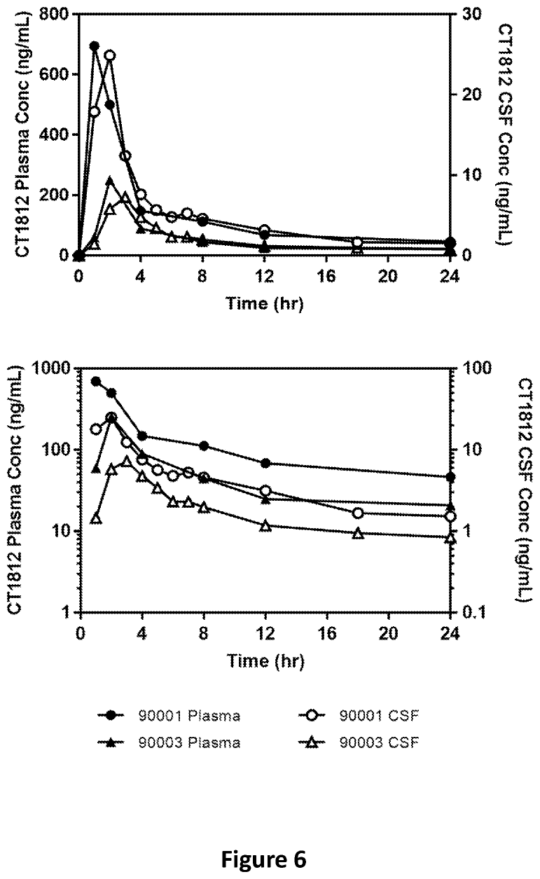

Interestingly, this robust drug-related increase in Ab oligomers over time is specific to oligomers. Percent change from baseline in Aβ 40 and 42 monomers were similar in all three patients (<50% increase). Aβ oligomer increased more than 200% in the two treated patients but did not increase in the placebo-treated patient. CSF Aβ oligomer levels were fit to a linear regression and the significance of the slope difference from zero was determined for each patient (FIG. 4 (measured by microimmunoelectrodes); FIG. 5 (measured by Western Blot)). A summary of plasma and CSF data are presented in Table 1 and FIG. 6.

TABLE 1
Summary of plasma and CSF data from NCT03522129

| | | Cₘₐₓ | tₘₐₓ | AUC₀₋ₗₐₛₜ | CSF/Plasma Ratio | |
|---|---|---|---|---|---|---|
| Matrix | Subject | (ng/mL) | (hr) | (hr*ng/mL) | Cₘₐₓ^{a} | AUC₀₋ₗₐₛₜ^{b} |
| Plasma | 90001 | 695 | 1.00 | 3160 | 0.0358 | 0.0381 |
| | 90003 | 250 | 2.00 | 1210 | 0.0291 | 0.0384 |
| CSF | 90001 | 24.9 | 2.00 | 120 | — | — |
| | 90003 | 7.27 | 3.00 | 46.4 | — | — |

^{a}based on Cₘₐₓ;
^{b}based on AUC₀₋ₗₐₛₜ
AUC₀₋ₗₐₛₜ: area under the curve from time 0 to the last measurable concentration;
Cₘₐₓ: maximum observed concentration;
tₘₐₓ: time of maximum concentration

Summary: The results of this study provide early proof of principle that we may be able to measure target engagement in Alzheimer's disease patients. The pharmacokinetics of this study suggest that there is an exposure dependent relationship with the rise in oligomers.

### Example 2: CT1812 Treatment Reduces Cognitive Decline

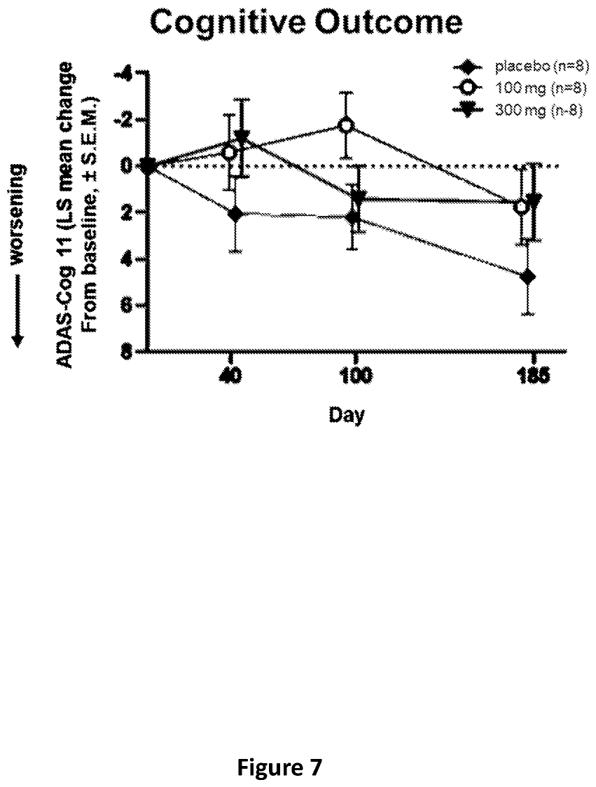

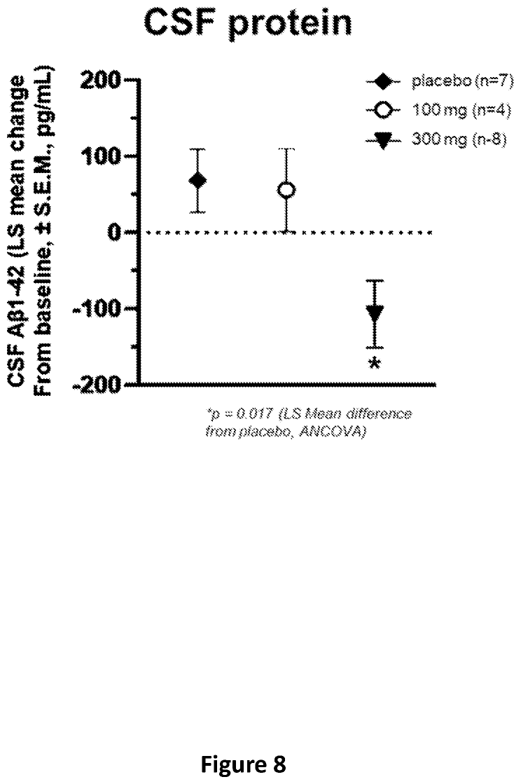

Patients treated for at least 6 months (185 days) with CT1812 were compared with patients who received placebo. Patients receiving CT1812 demonstrated a three-point difference Alzheimer's Disease Assessment Score (ADAS-COG)_between treated and untreated patients at day 185, representing a clinically meaningful magnitude of change suggesting a trend for improved cognitive outcomes (FIG. 7). Patients treated for at least 6 months (185 days) with CT1812 had lower Aβ protein (p=0.017) in treated versus placebo patients (FIG. 8).

We claim:

1. A method of reducing amyloid β monomer levels in a subject comprising administering to the subject a therapeutically effective amount of a compound of formula: 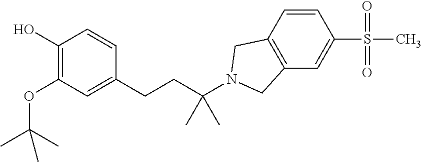 [SMILES: CC(C)(C)Oc1cc(CCC(C)(C)N2Cc3ccc(S(C)(=O)=O)cc3C2)ccc1O]
or a pharmaceutically acceptable salt thereof.

2. The method of claim 1, wherein the subject is a human.

3.-4. (canceled)

5. The method of claim 1, wherein the subject is a subject with Alzheimer's disease.

6. The method of claim 1, wherein the compound is administered for at least 6 months.

7. The method of claim 1, wherein the therapeutically effective amount is a total daily dose of about 10 mg to about 2000 mg administered for at least 6 months.

8. The method of claim 1, wherein the therapeutically effective amount is a total daily dose of about 300 mg administered for at least 6 months.

9. The method of claim 1, wherein the therapeutically effective amount is a total daily dose of 100 mg administered for at least 6 months.

10. The method of claim 1, wherein the amyloid β monomer is selected from Aβ 1-40 and Aβ 1-42.

11. The method of claim 1, wherein the method of reducing amyloid β monomer levels is relative to amyloid β monomer levels prior to administration of the compound.

12. The method of claim 1, wherein the amyloid β monomer levels are measured from cerebral spinal fluid, hippocampal interstitial fluid, plasma or a combination thereof.

13. The method of claim 1, wherein administering a therapeutically effective amount of the compound results in a reduction in the Alzheimer's Disease Assessment Score (ADAS-COG) score of a subject as compared to the ADAS-COG score of the subject before treatment.

14-26. (canceled)

27. A method of elevating amyloid β oligomer levels in a subject comprising administering to the subject a therapeutically effective amount of a compound of formula: 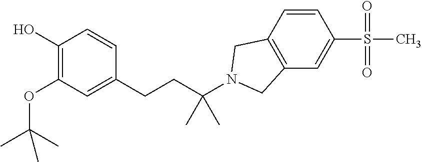 [SMILES: CC(C)(C)Oc1cc(CCC(C)(C)N2Cc3ccc(S(C)(=O)=O)cc3C2)ccc1O]
or a pharmaceutically acceptable salt thereof.

28. The method of claim 27, wherein the subject is a human.

29-30. (canceled)

31. The method of claim 27, wherein the subject is a subject with Alzheimer's disease.

32. The method of claim 27, wherein the compound is administered orally.

33. The method of claim 27, wherein the therapeutically effective amount is a total daily dose of about 10 mg to about 2000 mg.

34. The method of claim 27, wherein the therapeutically effective amount is a total daily dose of about 560 mg.

35. The method of claim 27, wherein the therapeutically effective amount is a total daily dose of about 300 mg.

36. The method of claim 27, wherein the therapeutically effective amount is a total daily dose of 100 mg.

37. The method of claim 27, wherein the method of elevating amyloid β oligomer levels is relative to amyloid β oligomer levels prior to administration of the compound.

38. The method of claim 27, wherein the amyloid β oligomer levels are measured from cerebral spinal fluid, hippocampal interstitial fluid, plasma or a combination thereof.

39. The method of claim 27, wherein the amyloid β oligomer levels are measured within 24 hours of CT1812 administration.

40.-53. (canceled)

54. A method of treating Alzheimer's disease in a subject comprising administering to the subject a therapeutically effective amount of a compound of formula: 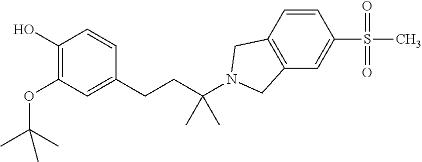 [SMILES: CC(C)(C)Oc1cc(CCC(C)(C)N2Cc3ccc(S(C)(=O)=O)cc3C2)ccc1O]
or a pharmaceutically acceptable salt thereof; wherein administering a therapeutically effective amount of the compound results in a change in levels of amyloid β in the subject.

55. The method of claim 54, wherein the subject is a human.

56. The method of claim 54, wherein the compound is administered for at least 6 months.

57. The method of claim 54, wherein the therapeutically effective amount is a total daily dose of about 10 mg to about 2000 mg administered for at least 6 months.

58. The method of claim 54, wherein the therapeutically effective amount is a total daily dose of about 300 mg administered for at least 6 months.

59. The method of claim 54, wherein the therapeutically effective amount is a total daily dose of 100 mg administered for at least 6 months.

60. (canceled)

61. The method of claim 54, wherein the levels of amyloid β are relative to amyloid β monomer levels prior to administration of the compound.

62. The method of claim 54, wherein the change in the levels of amyloid β is selected from a reduction in amyloid β monomers, an increase in amyloid β oligomers, or both.

63. The method of claim 54, wherein the amyloid β monomers is selected from Aβ 1-40 and Aβ 1-42.

64. The method of claim 54, wherein the amyloid β levels are measured from cerebral spinal fluid, hippocampal interstitial fluid, plasma or a combination thereof.

65. The method of claim 54, wherein administering a therapeutically effective amount of the compound results in a reduction in the Alzheimer's Disease Assessment Score (ADAS-COG) score of the subject as compared to the ADAS-COG score of the subject before treatment.

66. The method of claim 54, wherein administering a therapeutically effective amount of the compound results in an at least 3 point reduction in the Alzheimer's Disease Assessment Score (ADAS-COG) score of the subject as compared to the ADAS-COG score of the subject before treatment.
